(19) United States
(12) Patent Application Publication
(10) Pub. No.: US 2020/0405154 A1
(43) Pub. Date: Dec. 31, 2020

(54) APPARATUS AND METHOD FOR IN VIVO IMAGING OF SOFT AND HARD TISSUE INTERFACES

(71) Applicant: LIVE VUE TECHNOLOGIES INC., Kanata (CA)

(72) Inventors: Hassan Ghaderi MOGHADDAM, Kanata (CA); Jessie WEBER, Quebec (CA); Ozzy MERMUT, Woodbridge (CA)

(21) Appl. No.: 16/975,940

(22) PCT Filed: Feb. 28, 2019
(86) PCT No.: PCT/CA2019/050241

§ 371 Date: Aug. 26, 2020

Related U.S. Application Data
(60) Provisional application No. 62/636,355, filed on Feb. 28, 2018.

(51) Int. Cl. A61B 5/00; A61B 6/14; A61B 6/00

(52) U.S. Cl. CPC A61B 5/0088; A61B 5/0066; A61B 5/412; A61B 6/463; A61B 6/14; A61B 6/5247; A61B 5/0035

(57) ABSTRACT

An apparatus for determining a soft tissue/hard tissue interface including an optical coherence tomography system comprising at least one optical fiber for emitting and receiving detection light; and a probe including a gripping portion, and a signal delivery portion housing the at least one optical fiber, the delivery portion having a distal end portion, the distal end portion being bent such that an end face of the delivery portion generally faces an investigation region when in use. An apparatus for inspecting a soft tissue/hard tissue interface including an optical coherence tomography system comprising at least one optical fiber for emitting and receiving detection light; an near-infrared probing system operatively connected to the at least one optical fiber, the near-infrared probing system emitting at least a wavelength adapted for detecting a pre-determined condition.

### CROSS-REFERENCE

The present application claims priority to U. S. Provisional Patent Application No. 62/636,355, entitled “Apparatus And Method For In Vivo Imaging Of Soft And Hard Tissue Interfaces”, filed Feb. 28, 2018, the entirety of which is incorporated herein by reference.

### FIELD OF TECHNOLOGY

The present technology relates to the field of in vivo imaging, and more specifically but not exclusively to apparatus and methods for in vivo imaging of soft and hard tissue interfaces, such as the interface(s) of teeth, gum, and/or bone, and other structures through skin or epithelial layers.

### BACKGROUND

Dental x-rays are commonly used to image teeth, jaw and other mouth structures formed from bone, but are not generally capable of imaging gum or skin tissues. In many dental applications however, it is important to determine the condition of gums or skin tissues overlaying a patient's teeth, jaw, and other dental structures. In some cases, there is interest in determining the thickness of the tissues, as gum inflammation can be used to diagnose periodontal disease for which early and accurate diagnosis is important.

Other imaging modalities have been proposed for viewing bone structures through small layers of epithelial cells. These modalities are often implemented as large physical systems that cannot be used in the small, sensitive space of a patient's mouth. In some cases, additional CT-scans could be required to see certain structures, including some bone structures that cannot be imaged in standard dental x-rays due to resolution limits in x-ray imaging. Additional CT-scans can be time consuming, and generally expose a patient to additional radiation. Further, where there are metal structures in the area to be investigated, the metal can cause interference with CT-scans and impede production of images clear enough to be used for diagnosis.

As such, there remains a desire for apparatus and methods for imaging or differentiating in vivo soft and hard tissue structures, such as but not limited to dental and maxillofacial applications, and other applications in which imaging through epithelial layers is required such as to image organs or other internal structures.

### SUMMARY

It is an object of the present invention to ameliorate at least some of the inconveniences present in the prior art.

According to a first broad aspect of the present technology, there is provided an apparatus for determining or imaging a soft and hard tissue interface, such as an interface between gums and teeth in dental applications. The interfaces detected could relate to any number of structures, including but not limited to: gum, teeth, bone, dental implants, and/or dental prosthesis. The apparatus includes at least: an optical coherence tomography system comprising at least one optical fiber for emitting and receiving detection light; and a probe, including a gripping portion, and a signal delivery portion housing the at least one optical fiber, the delivery portion having a distal end portion, the distal end portion of the delivery portion being configured such that an end face of the delivery portion generally faces an investigation region when in use. The investigation region may be soft tissue/hard tissue interface such as the gum/tooth interface. In some embodiments, the distal end portion of the delivery portion is angled or bent with respect to the gripping portion. In some embodiments, at least the distal end portion is sized and shaped for insertion into an oral cavity of a patient. In certain embodiments, the apparatus can provide in-house measurements of a patient's bone, teeth and gums. In certain embodiments, the apparatus could be used to measure the depth or other structural or physical properties of periodontal pockets by imaging only, in place of a manual measurement performed by a dental professional. Therefore, in certain embodiments, a more accurate and reproducible measure of periodontal pocket depth and structural analysis can be achieved using the apparatus.

In some implementations of the present technology, the apparatus can further include a near-infrared probing system, which could provide either point, scanning, or imaging measurements. In certain embodiments, the apparatus can be used to detect functional characteristics of the tissue for example image inflammation of the soft tissue using biomarkers of inflammation which can be visualized using the near-infrared probing system. One such biomarker is cannabinoid receptor type 2 (CB2R), for example. Other biomarkers are possible, which may be used to image different soft tissue conditions. Therefore, the combination of the functional image data on gum inflammation using the near-infrared probing system together with the structural image data of the hard and soft tissue interface from the OCT and probe, may in certain embodiments, provide a useful diagnostic tool for a number of different dental and oral diseases and conditions.

According to an aspect, there is provided a method for measuring periodontal pocket depth, the method comprising imaging a soft tissue/hard tissue interface using the optical coherence tomography system and probe as described above. In certain embodiments, the method is performed by contacting the distal end portion of the probe with the soft tissue/hard tissue interface of a tooth of a patient. In certain embodiments, the angled distal end portion of the probe can facilitate reaching teeth at the back of the oral cavity. In certain embodiments, the method further comprises using an image processing algorithm to define at least one digital boundary at the soft tissue/hard tissue interface, and to measure a distance from the at least one digital boundary to a baseline boundary representative of a gum with no periodontal pockets, or with periodontal pockets having a depth within an acceptable range (e.g. normally less than 3 mm).

In certain implementations, the method further comprises applying a biomarker which can be detected by the near-infrared probing system to the soft tissue, and applying the near-infrared probing system to locate the biomarker. In certain embodiments, an imaged biomarker may be mapped to the OCT image for diagnosis or visualization of certain conditions.

According to another broad aspect of the present technology, there is provided a method for determining or imaging a soft and hard tissue interface, such as an interface between gums and teeth in dental applications (a dental interface), the method being implemented by a computer-implemented device. The method includes receiving a digitized version of a dental x-ray image of a given mouth region; receiving an optical coherence tomographic image of the given mouth region; aligning, by the computer-implemented device, the x-ray image and the tomographic image such that corresponding features of the given mouth region are superimposed; and presenting, on a visual interface of the computer-implemented device, the superimposed image with at least features of the given mouth region having been highlighted.

In some implementations, the method further includes visually highlighting, by the computer-implemented device, at least one anatomical feature of the given mouth region.

According to another broad aspect of the present technology, there is provided a multimodal system for determining or imaging a soft and hard tissue interface, such as an interface between gums and teeth in dental applications (a dental interface). The system comprises a computer-implemented device, such as a processor, for receiving a digitized version of a dental x-ray image of a given mouth region; receiving an optical coherence tomographic image of the given mouth region; aligning, by the computer-implemented device, the x-ray image and the tomographic image such that corresponding features of the given mouth region are superimposed; and presenting, on a visual interface of the computer-implemented device, the superimposed image with at least features of the given mouth region having been highlighted.

In some implementations, the near-infrared probing system is adapted to perform time-resolved functional imaging.

In some implementations, the pre-determined condition is at least one of infection; and inflammation.

In some implementations, the optical coherence tomography system provides structural information of the investigation region; and the near-infrared probing system provides functional information of the investigation region.

In some implementations, the optical coherence tomography system provides information related to variation in mechanical properties of the investigation region.

According to a yet further aspect of the present technology, there is provided an apparatus for determining a soft tissue/hard tissue interface, including an optical coherence tomography system comprising at least one optical fiber for emitting and receiving detection light; an near-infrared probing system operatively connected to the at least one optical fiber, the near-infrared probing system emitting at least a wavelength corresponding to infection and/or inflammation detection; and a probe, including: a gripping portion, and a signal delivery portion housing the at least one optical fiber, the delivery portion having a distal end portion, the distal end portion of the delivery portion being bent such that an end face of the delivery portion generally faces an investigation region when in use. Implementations of the present technology each have at least one of the above-mentioned object and/or aspects, but do not necessarily have all of them. It should be understood that some aspects of the present invention that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a diagram of a standard time-domain Optical Coherence Tomography setup of the prior art;

FIG. 2 is a diagram of a spatially-encoded Fourier-domain OCT system (SEFD-OCT);

FIG. 3 is a diagram of a frequency-swept-source-based OCT system (SS-OCT);

FIG. 4 is a perspective view of a probe according to one non-limiting embodiment of the present technology;

FIG. 5 is a perspective view the probe of FIG. 4, in use inspecting a mouth region;

FIG. 6 is a schematic illustration of one method according to one non-limiting embodiment of the present technology; and

FIG. 7 is a schematic diagram of a probe according to another non-limiting embodiment of the present technology.

It should be noted that the Figures may not be drawn to scale, unless otherwise noted.

### DETAILED DESCRIPTION

The present detailed description is intended to be only a description of illustrative examples of the present technology. The following description relates to ways of constructing and utilizing a system and a hand-held probe for detecting gum/bone and gum/teeth interfaces. Other uses of the system and probe are possible, such as detecting and imaging through skin, epithelial layers, and the like. Other ways of manufacturing and utilizing such a system or probe contemplated and this technology would encompass these other known ways and designs.

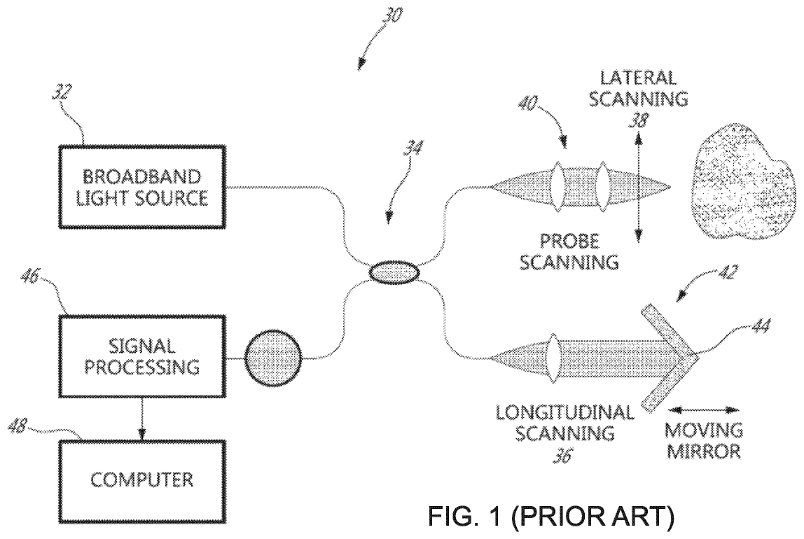

FIG. 1 shows an embodiment of a standard time-domain optical coherence tomography (OCT) system 30 using a low coherence light source 32 (typically a superluminescent LED or pulsed laser) and an interferometer configuration 34 for performing a longitudinal scanning 36 and a lateral scanning 38. As illustrated, an optical arrangement 40 is used for implementing the lateral scanning 38 while an optical arrangement 42 comprising a moving mirror 44 is used for implementing the longitudinal scanning 36. A detection system 46, including a signal processor, may be used in conjunction with a computer 48 for signal processing purposes.

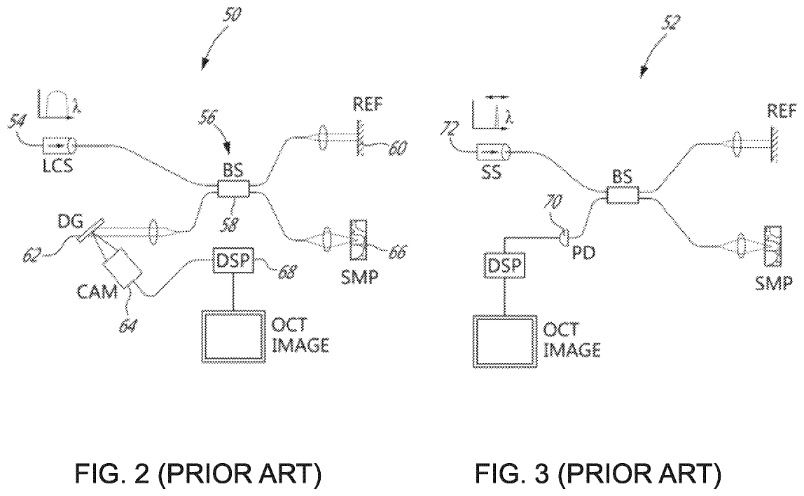

Newer designs, such as the systems 50 and 52 shown in FIGS. 2 and 3 respectively, involve detecting in the Fourier domain or using frequency-swept light sources to disband with the traditional time-pulsed requirement of the incident light emission. The system 50 comprises a low coherence source (LCS) 54, an interferometer sub-assembly 56 provided with a beamsplitter (BS) 58 and a reference mirror (REF) 60. The system 50 also comprises a diffraction grating (DG) 62 and a camera (CAM) 64 for detecting light back-scattered by the sample (SMP) 66. A digital signal processor (DSP) 68 is operatively connected to the camera 64 for providing an OCT image based on the back-scattered light. The system 52 of FIG. 3 uses a swept source (SS) 72 in place of the low coherence source 54 of FIG. 2 and a photodetector (PD) 70.

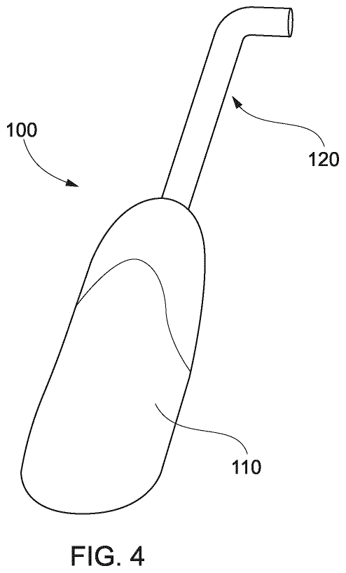

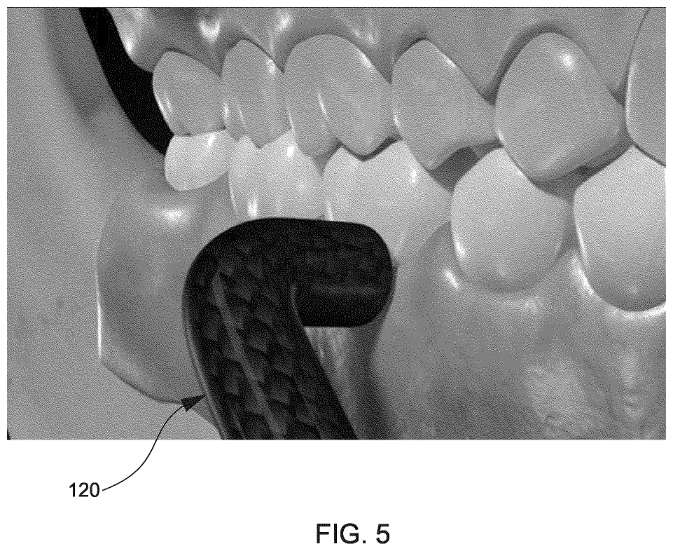

FIGS. 4 and 5 illustrate a hand-held probe 100 according to one non-limiting embodiment of the present technology. In some embodiments, the probe 100 may be controlled or connected to a machine, but is generally sized and adapted to be used in the hand of an operator.

The probe 100 is a forward-looking, sterilizable probe 100 that fits in the mouth of a patient. The probe 100 includes a gripping portion 110 for holding the probe 100. The gripping portion 110 is ergonomically sized and configured for holding in an operator's hand. The probe 100 could, in some embodiments, further include structures to enable the probe 100 to be safely set down or stored. In other embodiments, a detachable portion (not shown) of the probe 100 could be sterilizable.

The probe 100 is connected to an optical coherence tomography (OCT) system, such as those illustrated in FIGS. 1 to 3. In order to transmit light through the probe 100, the probe 100 includes a delivery portion 120. The delivery portion 120 houses an optical fiber or gradient index (GRIN) lens which is operatively connected to the OCT system. The delivery portion 120 extends outward from the gripping portion 110, but the specific form could depend on specifics of any given embodiment.

A distal end of the delivery portion 120 is generally flat or rounded, and exposes an optical element (generally a lens) housed therein. Depending on the specific implementation, the OCT system connected to the probe 100 can perform B- or C-OCT scans down each tooth to look at the interface of between a tooth and the surrounding gums, the gums and underlying bone, or both. Where the probe is used for different skin/epithelial applications, the distal end is shaped and oriented according to the specific investigation region.

A distal end portion of the delivery portion 120 is bent, as can be seen in the illustrations, such that the optical fiber can deliver (and receive) light signals in a portion of an oral cavity of a given subject. In other words, the distal end portion is angled with respect to the gripping portion. Where the probe 100 is used for different skin/epithelial application, the distal end portion is shaped and oriented according to the specific investigation region.

In the illustrated embodiment, the distal end portion is oriented at an angle about 90 degrees with respect to the gripping portion 110. It is contemplated that the angle between the distal end portion and the gripping portion 110 could be more or less than 90 degrees. In some embodiments, the delivery portion 120 may also be angled such that rear teeth can be more easily reached. As can be seen in FIG. 5, the distal end portion is angled such that the distal end of the delivery portion 120 generally faces the different structures of the oral cavity (teeth, gums, dental implants, bone, etc.) when the probe 100 is inserted into the oral cavity. In embodiments related to different applications, it is contemplated that the end portion could be oriented depending on the structures being detected, measured, or imaged.

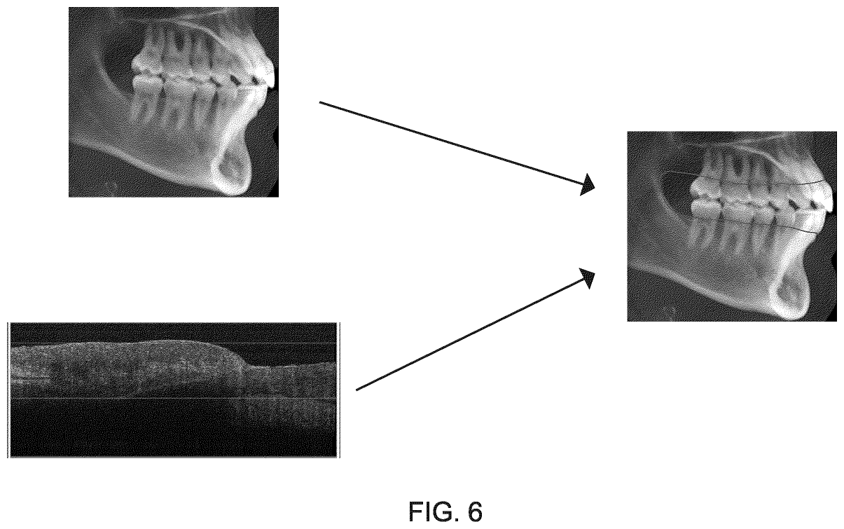

FIG. 6 schematically depicts a method according to another non-limiting embodiment of the present technology. The method is implemented by a computer-implemented device, which can include but is not limited to: a personal computer, a hand-held device, a smart phone, a cloud computing system, and a laptop computer.

The method includes receiving, by the computer-implemented device, a dental x-ray image. X-ray images produce images of hard tissue, such as bone and teeth in the dental context. In some implementations, the dental x-ray image could be a panoramic dental x-ray image, also referred to as a radiograph. The received image could be either a digital x-ray taken in digital form directly, or a standard film dental x-ray having been digitized (for example by scanning).

The method further includes receiving an OCT image of a mouth region that at least partially overlaps with the mouth region imaged in the dental x-ray received. In contrast to X-ray images, OCT images show soft tissues such as buccal and lingual tissues. In some implementations, the method can receive the OCT image from an earlier measurement. In some other implementations, the method can further include prompting and/or performing the OCT measurement upon receipt of the dental x-ray. In some implementations, the OCT image could be received from a system employing the hand-held probe 100 described above and illustrated in FIGS. 4 and 5.

In some embodiments, one imaging system could be used to produce both the X-ray and OCT images. As one example, a Panosonic™ scanner prototype including a metal oxide semiconductor camera for capturing a 10 mm by 10 mm area in use simultaneously with an OCT scanner could be used.

The method then includes aligning the two images, such that corresponding features of the mouth region imaged in the two images are properly aligned. The step of aligning the images could, in some implementations, include an algorithm for image mapping. In some other implementations, an operator of the computer-implemented device could provide indications for one or more features in the two images to be aligned.

The method then proceeds to presenting visually highlighted interfaces in the imaged mouth region by multimodal image fusion using a superposition of the (now-aligned) x-ray and OCT images. For example, the method can present to a user the x-ray image of the mouth region, with a false color image of the gums superimposed on the x-ray. In some embodiments, the combination of the X-ray image and the OCT image could produce a three-dimensional image.

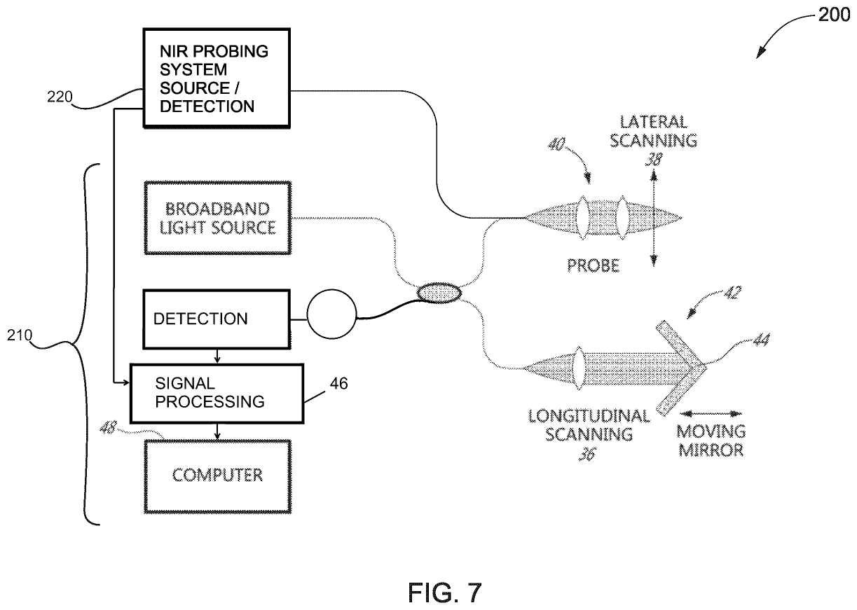

FIG. 7 schematically depicts another non-limiting embodiment of a device 200 according to the present technology. The device 200 includes an OCT system 210 operatively connected to the hand-held probe 100, as described above with regard to FIGS. 4 and 5. The device 200 further includes a near-infrared (NIR) probing system 220. The NIR system 220 can be implemented as a point detection system, line scanning system, and/or imaging system, depending on the specific application. The light source of the NIR system 220 is operatively connected to the probe 100, along with the OCT system 210, such that the signal processing module and computer can measure the NIR signals as well.

In some embodiments, the NIR system 220 is configured to emit light at a wavelength that is correlated with diseased gum tissues. For example, the NIR system 220 can be configured to emit light at a wavelength that is more highly absorbed by diseased gum tissue than healthy tissue, or light at a wavelength that is more highly reflected or scattered by diseased tissue. In such an implementation, the device 200 produces an image of the gum thicknesses and/or locations of gum/teeth/bine interfaces (from the OCT system 210), as well as a map of diseased gum tissue versus healthy gum tissue (from the NIR system 220). In some implementations, it is also contemplated that the NIR system 220 could be multi-spectral based (i.e. uses multiple different wavelengths of light for illuminating the investigation region). For example, the NIR system 220 could provide light at wavelengths corresponding to absorption or scattering for multiple different conditions.

In some embodiments, the OCT system 210 could also be used to perform elastography, measuring variation in mechanical properties, for the gum and/or skin tissue. The elastography measurements could be included as a surrogate or complimentary measurement of progression of periodontal disease, for example.

In some embodiments, the NIR system 220 could be adapted to perform time-resolved functional imaging. Time-resolved imaging is generally much more stable than intensity-based NIR detection. Depending on the embodiment, the time-resolved imaging could be done in both time-domain and frequency domain. By using time-resolved imaging for NIR detection, time resolved decay of intrinsic Hb or other chromophores can be detected in order to identify inflammation and/or infection. Moreover, time-resolved detection of the NIR probing system 220, whether in the time- or frequency-domain, could allow for better quantification of the images due to the independence of the chromophore concentration with respect to the detected signal, in contrast to intensity-based NIR probes. Such an image, identifying diseased or irregular tissue, can then be overlaid with an acquired structural map produced by the OCT imaging.

With use of the OCT system 210 and the NIR system 220, either in combination or taken alone, the system 200 provides one solution over at least one of the inconveniences typically encountered when utilizing CT or x-ray detection. Specifically, each of the OCT system 210 and the NIR system 220 can produce images or provide information during a procedure (i.e. in real time), in contrast to both CT or x-ray detection. As one non-limiting example, the present technology could allow for real time detection and analysis of dental interfaces during a periodontal treatment using the OCT system 210. Similarly, inflammation and/infection could be investigated in real time during a periodontal treatment, using the NIR system 220. Additionally, each of the OCT system 210 and the NIR system 220 can produce images with higher resolution than that available in x-ray or CT-scan detection, allowing for imaging of smaller structures. For instance, the apparatus 200 could be utilized to inspect structures of interest previously found in x-ray images.

Modifications and improvements to the above-described implementations of the present invention may become apparent to those skilled in the art. The foregoing description is intended to be exemplary rather than limiting. The scope of the present invention is therefore intended to be limited solely by the scope of the appended claims.

1. An apparatus for determining a soft tissue/hard tissue interface, comprising:
an optical coherence tomography system comprising at least one optical fiber for emitting and receiving detection light; and
a probe, comprising:
a gripping portion, and
a signal delivery portion housing the at least one optical fiber, the delivery portion having a distal end portion, the distal end portion being bent such that an end face of the delivery portion generally faces an investigation region when in use.

2. An apparatus for inspecting a soft tissue/hard tissue interface, comprising:
an optical coherence tomography system comprising at least one optical fiber for emitting and receiving detection light;
an near-infrared probing system operatively connected to the at least one optical fiber, the near-infrared probing system emitting at least a wavelength adapted for detecting a pre-determined condition; and
a probe, comprising:
a gripping portion, and
a signal delivery portion housing the at least one optical fiber, the delivery portion having an end portion, the end portion of the delivery portion being bent such that an end face of the delivery portion generally faces an investigation region when in use.

3. The apparatus of claim 2, wherein the near-infrared probing system is adapted to perform time-resolved functional imaging.

4. The apparatus of claim 2, wherein the pre-determined condition is at least one of:
infection; and
inflammation.

5. The apparatus of claim 2, wherein:
the optical coherence tomography system provides structural information of the investigation region; and
the near-infrared probing system provides functional information of the investigation region.

6. The apparatus of claim 2, wherein the optical coherence tomography system provides information related to variation in mechanical properties of the investigation region.

7. A method for inspecting a dental interface, the method being implemented by a computer-implemented device, the method comprising:
receiving a digitized version of a dental x-ray image of a given mouth region;
receiving an optical coherence tomographic image of the given mouth region;
aligning, by the computer-implemented device, the x-ray image and the tomographic image such that corresponding features of the given mouth region are superimposed; and
presenting, on a visual interface of the computer-implemented device, the superimposed image of the given mouth region.

8. The method of claim 7, further comprising:
visually highlighting, by the computer-implemented device, at least one anatomical feature of the given mouth region.
